(19) United States
(12) Patent Application Publication
(10) Pub. No.: US 2024/0159735 A1
(43) Pub. Date: May 16, 2024

(54) SYSTEM AND METHOD OF NOISE REDUCTION IN BLOOD PANELS

(71) Applicant: Ension, Inc., Butler, PA (US)

(72) Inventors: Patrick Cahalan, Fort Myers, FL (US); Linda Cahalan, Fort Myers, FL (US); Ali Hussain, Cape Coral, FL (US)

(21) Appl. No.: 18/535,664

(22) Filed: Dec. 11, 2023

Related U.S. Application Data
(63) Continuation-in-part of application No. 17/074,888, filed on Oct. 20, 2020.
(60) Provisional application No. 63/431,596, filed on Dec. 9, 2022.

(51) Int. Cl. G01N 33/50

(52) U.S. Cl. CPC G01N 33/50; G01N 2400/22; G01N 2400/40

(57) ABSTRACT

A system and method of noise reduction in a blood panel testing comprising the steps of: drawing blood samples from a patient; and testing blood samples within a blood analyzer configured for noise reduction wherein the blood contacting surfaces of the blood panel testing platform have been activated and have been subsequently subject to wet chemistry treatment including enhancing the blood contacting surface of the blood analyzer with a wet chemistry treatment including an aqueous solution having a strong oxidizing agent; adding a positively charged spacer molecule to the blood contacting surface with a wet chemistry treatment including an aqueous solution having a cationic polymer; and covalently immobilizing heparin to the blood contacting surface of the blood analyzer with a wet chemistry treatment including heparin.

### RELATED APPLICATIONS

The present application claims the benefit of U.S. Provisional Application Ser. No. 63/431,596 filed Dec. 9, 2023 titled “System and Method of Noise Reduction in Blood Panels” which is incorporated herein by reference.

The present application is a continuation in part of U.S. patent application Ser. No. 17/074,888 filed Oct. 20, 2020 titled “Engineered Heparin Bioactive Matrix for Clinical Application of Blood Contacting Surface and Method of Manufacturing the Same” which published Apr. 8, 2021 as publication number 2021/0100935 which application and publication are incorporated herein by reference.

U.S. patent application Ser. No. 17/074,888 claims the benefit of international patent application serial number PCT/US2018/061993 filed Nov. 20, 2018 and published Oct. 24, 2019 as WO 2019/203898, which application and publication are incorporated herein by reference.

International patent application serial number PCT/US2018/061993 claims the benefit of U.S. Provisional Application Ser. No. 62/660,804 filed Apr. 20, 2018 titled “Engineered Heparin Bioactive Matrix for Clinical Application of Blood Contacting Surface and Method of Manufacturing the Same” which is incorporated herein by reference.

### BACKGROUND OF THE INVENTION

### 1. Field of the Invention

This invention generally relates to a blood panels, and more particularly to noise reduction in blood panels.

### 2. Background Information

A blood test is a laboratory analysis performed on a blood sample that is usually extracted from a vessel in the arm using a hypodermic needle. Multiple tests for specific blood components, such as a glucose test or a cholesterol test, are often grouped together into one test panel called a blood panel or blood work. Blood tests are often used in health care to determine physiological and biochemical states, such as disease, mineral content, pharmaceutical drug effectiveness, and organ function. Blood tests are also used in drug tests to detect drug abuse.

Typical clinical blood panels include a basic metabolic panel or a complete blood count. A basic metabolic panel (BMP) is a blood test consisting of a set of seven or eight biochemical tests and is one of the most common lab tests ordered by health care providers. Outside the United States, blood tests made up of the majority of the same biochemical tests are called urea and electrolytes (U&E or “Us and Es”), or urea, electrolytes, creatinine (UEC or EUC or CUE), and are often referred to as ‘kidney function tests’ as they also include a calculated estimated glomerular filtration rate. The BMP provides key information regarding fluid and electrolyte status, kidney function, blood sugar levels, and response to various medications and other medical therapies. It is frequently employed as a screening tool during a physical exam. The basic metabolic panel is a simpler version of the comprehensive metabolic panel (CMP), which includes tests for liver function.

The version with seven tests is often referred to by medical professionals in the United States as the “CHEM-7”, or “SMA-7” (Sequential Multiple Analysis-7). The seven parts of a CHEM-7 are tests for: Four electrolytes (sodium (Na⁺) potassium (K⁺) chloride (Cl⁻) and bicarbonate (HCO₃⁻) or CO₂ blood urea (BU)), blood urea nitrogen in the U.S., creatinine, and glucose. These levels, taken as a set, can be rapidly performed to indicate several common acute conditions requiring immediate specific medical treatment, such as dehydration/hypovolemia, water intoxication (which can present with similar symptoms to dehydration but requires the opposite treatment), diabetic shock (either ketoacidosis, hyperglycemia or hypoglycemia), congestive heart failure, kidney failure or liver failure, various substance overdoses or adverse reactions, and others. A Chem-7 is thus a vital tool when attempting to stabilize a patient. Calcium (Ca²⁺) is often considered part of the BMP, though, by definition, it is not part of the CHEM-7. A basic metabolic panel including calcium is sometimes colloquially referred to as a “CHEM-8”. Calcium, as an alkaline earth metal, is also an electrolyte, but abnormalities are more commonly associated with malnutrition, osteoporosis, or malignancy, especially of the thyroid.

An assay is an investigative or analytic procedure in laboratory medicine, mining, pharmacology, environmental biology and molecular biology for qualitatively assessing or quantitatively measuring the presence, amount, or functional activity of a target entity. The measured entity is often called the analyte, the measurand, or the target of the assay. The analyte can be a drug, biochemical substance, chemical element or compound, or cell in an organism or organic sample. An assay usually aims to measure an analyte's intensive property and express it in the relevant measurement unit (e.g. molarity, density, functional activity in enzyme international units, degree of effect in comparison to a standard, etc.). If the assay involves exogenous reactants (the reagents), then their quantities are kept fixed (or in excess) so that the quantity and quality of the target are the only limiting factors. The difference in the assay outcome is used to deduce the unknown quality or quantity of the target in question. Some assays (e.g., biochemical assays) may be similar to chemical analysis and titration. However, assays typically involve biological material or phenomena that are intrinsically more complex in composition or behavior, or both. Thus, reading of an assay may be noisy and involve greater difficulties in interpretation than an accurate chemical titration. On the other hand, older generation qualitative assays, especially bioassays, may be more gross and less quantitative (e.g., counting death or dysfunction of an organism or cells in a population, or some descriptive change in some body part of a group of animals). There is a need for medical assays to improve the “signal of interest” to obtain better quantitative results.

Assays have become a routine part of modern medical, environmental, pharmaceutical, and forensic technology. In medicine, an assay is an analysis used to determine the presence of a particular substance and its concentration. Thus, for example, an assay may be conducted on a vaccine, to determine its effectiveness or potency in preventing illness. There are several types of medical assays. In addition to the potency and presence assays already mentioned, medicine uses a number of assays for determining the effectiveness of the immune system, exposure to certain viruses including those leading to AIDS, detecting certain sexually transmitted diseases, and working with thyroid hormones. Medical assays testing blood are considered blood panels within the present application.

Patients are often nervous about blood tests and will inquire about how much blood is to be taken for and given blood panel. The amount needed depends on the particulars of the blood panel being performed upon the patient. Several tests can be done on the same sample of blood. It is not uncommon for a patient having about 30 ml of blood (about six medicine teaspoons) taken. For comparison, about 500 ml of blood are given by blood donors during each donation. Additionally it is noted that some blood tests require several samples taken over a period of time. For example, this may be done to check how the patient is responding to something.

A blood chemistry analyzer is used in performing the blood panels for patients. Blood analyzers are used by hospitals, medical labs, forensic labs, and even by people at home. As suggested above a blood chemistry analyzer may be used to test for many things, such as blood cell counts, therapeutic drug monitoring, illegal drug use, blood typing, protein analysis, checking thyroid function, checking for the presence of antibodies, and, when used by patients at home, for glucose or cholesterol monitoring. Thus there are several types of tests used by blood analyzers, including cell counters for doing the blood counts, immunoassays for detecting antibodies, tests for ions that measure voltage differences, and tests for the presence of enzymes that detect enzyme activity by a physical change in the sample.

There remains a need for improving blood panels and reducing the noise in blood panels.

### SUMMARY OF THE INVENTION

One aspect of the present invention provides A system and method of noise reduction in a blood panel testing comprising the steps of: drawing blood samples from a patient; and testing blood samples within a blood analyzer configured for noise reduction wherein the blood contacting surfaces of the blood panel testing platform have been activated and have been subsequently subject to wet chemistry treatment including enhancing the blood contacting surface of the blood analyzer with a wet chemistry treatment including an aqueous solution having a strong oxidizing agent; adding a positively charged spacer molecule to the blood contacting surface with a wet chemistry treatment including an aqueous solution having a cationic polymer; and covalently immobilizing heparin to the blood contacting surface of the blood analyzer with a wet chemistry treatment including heparin.

One aspect of the invention provides A method of noise reduction in a blood panel testing platform comprising the steps of: activating the blood contacting surfaces of the blood panel testing platform; performing wet chemistry treatments on each blood contacting surface of the blood panel testing platform including enhancing the blood contacting surface of the blood panel testing platform with a wet chemistry treatment including an aqueous solution having a strong oxidizing agent; adding a positively charged spacer molecule to the blood contacting surface with a wet chemistry treatment including an aqueous solution having a cationic polymer; and covalently immobilizing heparin to the blood contacting surface of the blood panel testing platform with a wet chemistry treatment including heparin.

The various embodiments and examples of the present invention as presented herein are understood to be illustrative of the present invention and not restrictive thereof and are non-limiting with respect to the scope of the invention.

### BRIEF DESCRIPTION OF THE DRAWINGS

FIG. 1 is a schematic view of a manufacturing facility for forming a blood panel testing platform having an engineered heparin bioactive matrix of blood contacting surface in accordance with one aspect of the present invention.

### DESCRIPTION OF THE PREFERRED EMBODIMENTS

The noise reduction in blood panels according to the invention is configured to minimize background noise in testing by, in part, minimizing the variance of the measured activated species.

As used herein a blood chemistry analyzer is a medical device used to analyze blood samples and perform qualitative and quantitative analysis of the formed elements in the blood, providing relevant information, in other words it is a medical device used for performing the blood panels for patients. As detailed above a blood chemistry analyzer may be used to test for many things, such that there are several types of tests used by blood analyzers and there can be a variety of blood analyzers. The blood panel also utilizes conventional elements to obtain the blood sample and format the sample for the analyzer such as vials, syringes, cartridges, which can vary from analyzer to analyzer and are collectively referenced herein as blood receiving components. The blood analyzer and the blood receiving components are collectively referenced herein as the blood panel testing platform 50.

Specifically, within the present invention the blood contacting surfaces of the blood analyzer and the blood receiving components, again collectively referenced as the blood panel testing platform 50, has a coefficient of variance of less than 15% of the measured activated species. The blood panel testing platform 50 according to the invention is configured to minimize background noise by, in part, minimizing the effect of the surface of the platform 50 on the blood. The preferred structure is to employ a surface treatment of an engineered heparin bioactive matrix onto all the surfaces within the platform 50, resulting in a covalently bonded heparin surface throughout the blood contacting portion of the blood panel testing platform 50. The surface treatment outlined herein are suitable for the present platform 50 and yields a bioactivity level of 0.6+/−0.2 IIa deactivation/cm².

It is noted that, as used in this specification and the appended claims, the singular forms “a,” “an,” and “the” include plural referents unless expressly and unequivocally limited to one referent. For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques. All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements. The various embodiments and examples of the present invention as presented herein are each understood to be non-limiting with respect to the scope of the invention.

As used in the following description and claims, the following terms have the indicated meanings: The terms “on”, “appended to”, “affixed to”, “bonded to”, “adhered to”, or terms of like import means that the designated item, e.g., a coating, film or layer, is either directly connected to (superimposed on) the object surface, or indirectly connected to the object surface, e.g., through one or more other coatings, films or layers (superposed on). The terms “attach”, “couple”, and “link” refer to securing a material or biomolecule to a substrate, for example, by chemical covalent or ionic bonding, such that the coating or biomolecule is immobilized with respect to the substrate. The term “biomolecule” refers to a biologically active molecule. A “biocompatible” material does not generally cause significant adverse reactions (e.g., toxic or antigenic responses) in the body, whether it degrades within the body, remains for extended periods of time, or is excreted whole (see ISO 10993 as of Dec. 9, 2022). Ideally, a biocompatible material will not induce undesirable reactions in the body as a result of contact with bodily fluids or tissue, such as infection, coagulation, tissue death, tumor formation, allergic reaction, foreign body reaction (rejection) or inflammatory reaction. A “blood compatible” material is one that will not induce undesirable reactions in the body as a result of contact with blood, such as blood clotting or infection. A blood compatible material is understood to be biocompatible.

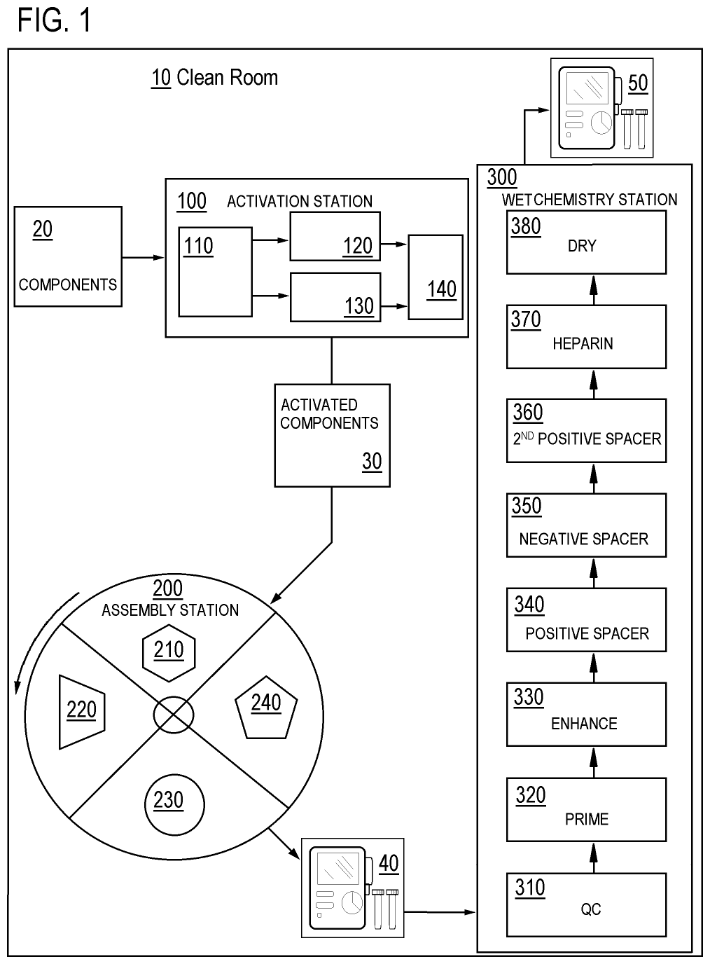

FIG. 1 is a schematic view of a manufacturing facility for forming a blood panel testing platform 50 for clinical application having an engineered heparin bioactive matrix of blood contacting surface in accordance with the present invention. The present invention is related to an engineered heparin bioactive matrix on a blood contacting surfaces of blood panel testing platform 50 as described below, but the present method and manufacturing system may be used for other biomolecules.

An engineered heparin bioactive matrix, or any engineered bioactive matrix within the meaning of this application refers to a substrate which has undergone surface treatment in accordance with engineering principles of maintaining homeostasis (See ISO 10993-4), and specific to blood contacting surfaces, maintaining hemostasis, namely maintaining the ability of the blood to maintain normal function as determined or verified by measuring the molecular and cellular elements of the blood maintaining within accepted clinical parameters. The engineered heparin bioactive matrix of blood contacting surface in accordance with present invention yields clinically relevant Physiological bio-functionality (bioactivity) which is stable over a long period of time.

In summary, the present invention provides a method of manufacturing a blood panel testing platform 50 for clinical application having an engineered heparin bioactive matrix on a blood contacting surface in which components 20 have surface activation of select surfaces at an activation station 100; the activated components 30 of the platform 50 are forwarded to an assembly station 200, and the assembled platform 40 is forwarded to a wet chemistry station 300 for application of having an engineered heparin bioactive matrix on a blood contacting surface to form the finished blood panel testing platform 50. The essential steps include a) activating a blood contacting surface of at least one component of a blood panel testing platform 50 via one of plasma treatment 120 or gas activation 130; b) assembling the platform 40 at station 200; c) setting up the assembled platform 40 for wet chemistry (310, 320) in which wet chemistry treatments contacts the blood contacting surfaces of the blood panel testing platform 50; d) enhancing at step 330 at least the blood contacting surface of the platform 50 with a wet chemistry treatment including an aqueous solution having a strong oxidizing agent; e) adding a positively charged spacer molecule at step 340 to at least the blood contacting surface of the platform 50 with a wet chemistry treatment including an aqueous solution having a cationic polymer; and f) covalently immobilizing heparin at step 370 to at least the blood contacting surface of the platform 50 with a wet chemistry treatment including heparin.

The manufacturing process of the present invention is configured for being performed within a single clean room 10. Clean room 10 is an enclosed and environmentally-controlled space in which temperature, humidity, pressure and contaminant levels are kept within defined limits. The controlled environment provided by the clean room 10 helps to ensure that blood panel testing platforms 50 remain under controlled contamination levels throughout the production process. The single clean room 10 for the process of the present invention is beneficial for the production of effective blood panel testing platforms 50. Details for the construction and operation of the clean room 10 are set forth in ISO 14644 (as of Dec. 9, 2022).

The components 20 forming the blood panel testing platform 50 are brought into the clean room 10 and to an inspection station 110 at the activation station 100. The process of the present invention may, likely, be performed on existing blood panel testing platforms to improve operation thereof. Where the blood panel testing platform 50 is formed upon an existing blood panel testing platform, the components 20 may be obtained by disassembling the existing platform for inspection and activation.

Following the inspection, the individual components 20 typically goes onto the activation via plasma treatment 120 or gas activation 130, or may go to a cleaning step if the inspection identifies an issue, or a component 20 may be discarded and replaced if a cleaning cannot remedy the problem with the component 20. Any component 20 of the platform 50 must pass its own inspection. It is possible that some components 20 will undergo a pre-cleaning before the clean room to assure that the component 20 is beginning from a desired state, and such pre-cleaning may be useful if there is variability from manufacturing or multiple vendors.

The components 20 passing inspection at step 110, which may be cleaned and inspected again, move onto activating a blood contacting surface of at least one component 20 of the platform 50 via one of plasma treatment 120 or gas activation 130.

Plasma treatment 120 take place within a plasma chamber containing electrodes, across which a voltage is applied. The chamber may be partially evacuated. The plasma treatment of the present invention may be with propene, oxygen, siloxane (hexamethyldisiloxane-HMDSO and/or tetramethyldisiloxane-TMDSO) or acrylic acid, in which a stream of the relevant gas is fed into the chamber. When a high frequency voltage is applied between the electrodes, current flows into the chamber, forming a plasma, which is a glowing electrical discharge within the gas. Reactive chemical species are formed in this electrical discharge. Plasma treatment 120 has certain advantages over gas treatment 130 and may be preferable for polycarbonate substrates.

Gas activation 130 within the meaning of this application is ozone activation or ozone treatment also known as UV ozone treatment. Gas activation 130 may be done at ambient pressures. Gas activation 130 may be preferable for polypropylene (PP), polyurethane (PU) polyethylene (PE) and silicone substrates.

Following surface activation via one of plasma treatment 120 or gas activation 130, each component 20 is moved to performance testing 140 in which representative components 20 are inspected and tested using conventional testing such as one of FTIR, SEM Contact angle and XPS. Following passing of the performance testing 140 the activated components 30 are forwarded to the assembly station 200. The record of the performance testing 140 is maintained to establish a quality review record of the production of the blood panel testing platform 50, or for a batch of the finished blood panel testing platforms 50.

The activated components 30 are assembled into the platforms 40 at station 200 having four assembly or operations stations 210, 220, 230 and 240 shown for reference. There may be more or less stations depending on the assembly or reassembly operations. The assembly station 200 may be efficiently formed in a compact arrangement as an index able turntable, or a stationary platform with a central pick and place robot. The process of the invention remains substantially the same if workers manually advance the components along through the station 200. Activated components 30 are assembled at the station 200 to form the assembled platform 40 which can be moved to the wet chemistry station 300.

The assembled platform 40 with some activated surfaces is moved to the wet chemistry station 300 for wet chemistry treatments which contacts and treats at least the blood contacting surfaces of platform 50. The first step is setting up the assembled device 40 for wet chemistry with in-line quality control at step 310. The present invention contemplates a batch process for manufacturing multiple platforms 50 simultaneously.

The platforms 40 must be set up such that all platforms 40 receive uniform treatment to assure uniform finished platforms 50. In this context parallel set ups rather than serial alignment of platforms 40 is preferred, as serial alignment of platforms 40 can yield lower treatment yields/levels for downstream devices and unacceptable lack of uniformity between platforms 50. The set up includes coupling the platforms 40 to source of wet chemistry treatments.

Additionally the present invention provides for in-line quality control through the visual staining of sacrificial inline tubing section, as desired. In other words the present method includes additional inline sacrificial tubing for processing platform 50 (or set of platforms 50). The tubing will be selectively removed and stained to determine treatment effectiveness and provide a verifiable record of quality control. The initial sacrificial tubing section of step 310 represents the baseline before wet chemistry treatments have started on the connected platforms 40 for creating a quality review record of the process for the platform 50 (as the quality review is determined to be needed).

The next step 320 is a water priming step which is also used for checking for leaks or undesired blockage in the set up to verify the set up arrangement. The water priming is accomplished at ambient or room temperature and is also utilized for calculating priming and treatment volumes for the coupled platform 40 and treatment apparatus.

Following the priming step of 320, the priming volume is removed from the platform 40 and the process proceeds to the enhancing step 330 for enhancing at least the blood contacting surface with a wet chemistry treatment including an aqueous solution having a strong oxidizing agent, preferably Ammonium persulfate (NH₄)₂S₂O₈. Specifically, [1-15%] (NH₄)₂S₂O₈ in (deionized) DI H₂O. The ammonium persulfate will enhance the activated and non-activated component surfaces of the medical device 40 as ammonium persulfate assists in activation of PVC and polycarbonate which may represent other components of a tubular oxygenator other than the fiber bundle that is treated as described above. The Enhancing wet chemistry treatment of step 330 is preferably at an elevated temperature of at least 50° C., preferably 60° C.-65° C., and contacts the blood contacting surface of the platform 40 for a treatment time of 5-45 minutes, preferably 15-30 minutes. The enhancing step 330 may then remove the treating aqueous solution and further includes a multiple non-recirculating rinse of about 3× priming volume of DI water at room temperature.

Following the enhancing step 330 is a step 340 of coupling a positive spacer, namely adding a positively charged spacer molecule to at least the blood contacting surface with a wet chemistry treatment including an aqueous solution having a cationic polymer. Preferably this cationic polymer solution is a 0.001 to 0.1% w aqueous solution of positively charged spacer polyethylenimine (PEI) molecule with cross-linker, preferably crotonaldehyde (CH₃CH═CHCHO) in buffer at a pH of about PH9. The cross linker crotonaldehyde may be present in concentrations of about 100-5001.1/liter solution. The positive spacer wet chemistry treatment of step 340 is preferably ambient or room temperature and is maintained on the blood contacting surfaces of the platform(s) 40 for a treatment time of 5-45 minutes, preferably 15-30 minutes. The positive spacer wet chemistry treatment of step 340 may then remove the treating aqueous solution and further includes a non-recirculating rinse of about 3× priming volume of DI water at room temperature.

The positive spacer wet chemistry treatment of step 340 then may include a quality control check of taking an in-line sacrificial tube section and stain the section and maintaining the results as a record of the wet chemistry process. This section of step 340 can be objectively compared to the baseline section taken in step 310. The quality review steps discussed here can be accomplished with every process for every platform 50, but in typical practice it may be accomplished on selected platforms 50 (often a given total number of quality reviews, say 10-30, at random intervals for every 1000 units produced). The main advantage of the quality review of the present invention is that it provides objective analysis without sacrificing production units of platforms 50. The final platforms 50 formed in manufacturing may have a certain number sacrificed for other regulatory requirements which this quality control paradigm might not satisfy and the present quality review paradigm allows the present methodology to go beyond the minimum reviews required without sacrificing additional platforms 50.

Following the positive spacer wet chemistry treatment of step 340 is a step 350 of coupling a positive spacer, namely adding a negatively charged spacer molecule to at least the blood contacting surface with a wet chemistry treatment including an aqueous solution having an anionic polymer. Preferably this anionic polymer solution is a 0.001 to 0.1% w of negatively charged spacer such as Dextran sulfate in saline solution pH 3. The negative spacer wet chemistry treatment of step 350 is preferably at an elevated temperature of at least 50° C., preferably 60° C.-65° C., and is maintained on the blood contacting surfaces of the platform(s) 40 for a treatment time of 5-45 minutes, preferably 15-30 minutes. The negative spacer wet chemistry treatment of step 350 then includes a non-recirculating rinse of about 3× priming volume of DI water at room temperature. The negative spacer wet chemistry treatment of step 350 then may, if desired, include a quality control check of taking an in-line sacrificial tube section and stain the section and maintaining the results as a record of the wet chemistry process.

Following the negative spacer wet chemistry treatment of step 350 is a step 360 of coupling a 2^{nd} positive spacer, namely adding a positively charged spacer molecule to at least the blood contacting surface with a wet chemistry treatment including an aqueous solution having a cationic polymer. Preferably this cationic polymer solution is a 0.001 to 0.1% w aqueous solution of positively charged spacer (PEI) molecule (with no cross-linker) in DI water at a pH of about PH9. The 2^{nd} positive spacer wet chemistry treatment of step 360 is preferably ambient or room temperature and is maintained on the blood contacting surfaces of the platforms 40 for a treatment time of 5-45 minutes, preferably 15-30 minutes. The 2^{nd} positive spacer wet chemistry treatment of step 360 then may include removal of the treating solution and a non-recirculating rinse of about 3× priming volume of DI water at room temperature. The 2^{nd} positive spacer wet chemistry treatment of step 360 then may include a quality control check, if desired, of taking an in-line sacrificial tube section and stain the section and maintaining the results as a record of the wet chemistry process.

Following the 2^{nd} positive spacer wet chemistry treatment of step 360 is a step 370 of covalently immobilizing heparin to at least the blood contacting surface with a wet chemistry treatment including heparin. Preferably the Heparin covalent immobilization wet chemistry treatment is 0.1 to 2 mg/ml, and preferred 0.4 to 1.4 mg/ml, deaminated heparin, in Sodium chloride solution at pH 3.5-4.5. Additionally, sodium cyanoborohydrite may be added in amounts of 0.01 to 0.1% w to the heparin solution for the wet chemistry around 5 to 15 minutes prior to circulation of the heparin solution wet chemistry treatment of step 370. The heparin wet chemistry treatment of step 370 is preferably at an elevated temperature of at least 50° C., preferably 60° C.-65° C., and is maintained on the blood contacting surfaces of the platform (s) 40 for a treatment time of 60-180 minutes, preferably 90-150 minutes. The heparin wet chemistry treatment of step 370 then includes removing the treatment solution and a non-recirculating 1^{st} rinse of about 3× priming volume of DI water at room temperature.

Following the initial water rinse the heparin wet chemistry treatment of step 370 may include a sodium chloride solution (0.2-0.8M Sodium Chloride) rinse which is maintained on the blood contacting surfaces of the platform(s) 40 at ambient conditions for 5-20 minutes, and then this is followed by non-recirculating 2^{nd} water rinse of about 3× priming volume of DI water at room temperature.

The heparin wet chemistry treatment of step 370 then may, if desired, include a quality control check of taking an in-line sacrificial tube section and stain the section and maintaining the results as a record of the wet chemistry process.

Following the heparin wet chemistry treatment of step 370 is a drying step 380 that may include a pressured gas flush of the platform 40 with an inert gas such as argon or nitrogen or air for 5 to 10 minutes, followed by clean filtered dry air a period of 8 to 24 hours to complete dehydration.

The drying of step 380 then may, if desired, include a quality control check of taking an in-line sacrificial tube section and stain the section and maintaining the results as a record of the wet chemistry process, and this may be followed by testing or measuring of heparin bioactivity in the devices 50.

An alternative to the above process is not utilizing the negative spacer of step 350 and the 2^{nd} positive spacer of step 360. In this engineered heparin matrix only a single PEI spacer is present. The process may be described as the same as above but not including the cross linker in step 340 and thereafter proceeding directly to the heparin wet chemistry treatment of step 370 while skipping steps 350 and 360, an alternative explanation is that the process skips from the enhancing step 330 to the second positive spacer step 360 Skipping steps 340-350. Both descriptions of this alternative arrangement are accurate and simply define a single positive spacer.

The manufacturing method of the present invention is universally applicable in that it defines a method of manufacturing platforms 50 with the ability to meet standard physiologically significant bioactivity with increased stability for clinical applications on a collection of substrates in a variety of types of existing platforms.

The ability to covalently immobilize heparin on all the blood contacting surfaces, including those of dis-similar material, is believed to be significant. It is understood that even a relatively small untreated area in the interior blood contacting area of the blood panel testing platform 50 may hinder the ability to obtain statistically significant comparative data with materials or coatings to be tested. The interior of the blood panel testing platform 50, treated as noted above, provides uniform treatment of all blood contacting surfaces even though there are different materials. Thus, assuring minimal background noise, and maximum ability to detect signals of interest from even small sample sizes.

The application of the identified bioactive surface technology is to improve the signal-to-noise ratio of blood panels or chemistry assays (e.g., the tests involved in a complete blood count panel) using the platform 50. By decreasing the background noise using the application of the identified bioactive surface technology on the blood-contacting surfaces of analyzer components in the platform 50, the signal-to-noise ratio is significantly increased. With a greater signal-to-noise ratio, the sample of blood could be decreased, the throughput of testing increased, and costs reduced. This is important as one of the most common problems in specimen collection in conventional platforms is the submission of an insufficient volume of specimen for testing. The laboratory often sends out a report marked QNS (quantity not sufficient), and the patient has to be called back for a repeat collection at an inconvenience to the patient and to the physician. Further, the ability of the platform 50 to decrease the needed sample size may enable moving from the relatively large volumes associated with traditional venipuncture (e.g., test tubes) to smaller samples that might be obtained from “finger pricks”.

The use of the platform 50 is conventional except for the reduction of noise and the ability to utilize a smaller sample for specific testing. The use of platform 50 contributes more precise vital information about a patient's health. Correct diagnostic and therapeutic decisions rely, in part, on the accuracy of test results. The platform improves testing results by reducing noise.

The process of obtaining blood panels with the platform 50 begins with collecting blood specimens within blood receiving devices that are treated as discussed above. In all settings in which specimens are collected and prepared for testing, laboratory and health care personnel should follow current recommended sterile techniques, including precautions regarding the use of needles and other sterile equipment.

Prior to each collection, professional should review the appropriate test description, including the specimen type indicated, the volume of the specimen, the procedure, the collection materials, patient preparation, and storage and handling instructions. As is conventional the patient should be provided, in advance, with appropriate collection instructions and information on fasting, diet, and medication restrictions when indicated for the specific test.

The testing, laboratory and health care personnel must verify the patient's identification as proper identification of specimens is extremely important. All primary specimen containers must be labeled, preferably with at least two identifiers at the time of collection. Examples of acceptable identifiers include (but are not limited to): patient's name (patient's first and last name exactly as they appear on the test request form), date of birth, hospital number, test request form number, accession number, or unique random number. Preferably all specimens should be labeled in the presence of the patient. Process and store the specimen(s) as required. Appropriate storage and handling are necessary to maintain the integrity of the specimen and, consequently, the test results. Material treated in accordance with the present invention for specimen collection can improve the quality of the specimens over prior materials.

The testing, laboratory and health care personnel have been advised to label a specimen correctly and provide all pertinent information required on the test request form and to submit a quantity of specimen sufficient to perform the test and avoid a QNS (quantity not sufficient), as indicated in the test requirements. As noted the use of the platform 50 can minimize this issue by obtaining effective results with less quantities.

Much of the instruction and training given to the testing, laboratory and health care personnel is not altered with the platform 50. For example, they will be advised to separate serum from red cells within two hours of venipuncture, to mix by inverting specimen with additive immediately after collection, to allow specimens collected in a clot tube (eg, red-top or gel-barrier tube) to clot before centrifugation, to avoid hemolysis: red blood cells broken down and components spilled into serum, to avoid lipemia: cloudy or milky serum sometimes due to the patient's diet.

Regarding plasma preparation, the instruction and training given to the testing, laboratory and health care personnel is not altered with the platform 50. The most common considerations in the preparation of plasma: collect specimen in additive indicated in the test requirements, mix specimen with additive immediately after collection by inverting 5-10 times, avoid hemolysis or red blood cell breakdown, fill the tube completely, thereby avoiding a dilution factor excessive for total specimen volume (QNS), separate plasma from cells within two hours of venipuncture or as indicated in the test requirements, label transport tubes as “plasma”, and indicate type of anticoagulant (eg, “EDTA,” “citrate,” etc.).

Once a specimen reaches the laboratory, the platform 50 operates in a conventional fashion. Most testing procedures on most conventional platforms are performed in 1-10 minutes. High-throughput labs have implemented automation of their hematology workflow and have integrated autoloader capacity in their platforms, and the platform 50 can utilize any desired automatic loading. The main operational difference is noise reduction in the results. The laboratory may take such noise reduction advantages of the platform 50 into account in setting the sample volume requirements for given tests and the platform 50 may have a reduction in the recommended volume for any test, as discussed above.

The present invention is not limited to the representative examples discussed above but is defined by the attached claims and equivalents thereto.

What is claimed is:

1. A method of noise reduction in a blood panel testing platform comprising the steps of:
activating the blood contacting surfaces of the blood panel testing platform;
performing wet chemistry treatments on each blood contacting surface of the blood panel testing platform including
i) enhancing the blood contacting surface of the blood panel testing platform with a wet chemistry treatment including an aqueous solution having a strong oxidizing agent;
ii) adding a positively charged spacer molecule to the blood contacting surface with a wet chemistry treatment including an aqueous solution having a cationic polymer; and
iii) covalently immobilizing heparin to the blood contacting surface of the blood panel testing platform with a wet chemistry treatment including heparin.

2. The method of noise reduction in a blood panel testing platform according to claim 1 wherein the step of enhancing at least the blood contacting surface with a wet chemistry treatment including an aqueous solution having a strong oxidizing agent includes ammonium persulfate.

3. The method of noise reduction in a blood panel testing platform according to claim 2 wherein the step of adding a positively charged spacer molecule to at least the blood contacting surface with a wet chemistry treatment including an aqueous solution having a cationic polymer utilizes PEI as the spacer molecule.

4. The method of noise reduction in a blood panel testing platform for clinical application according to claim 1 wherein the step of adding a positively charged spacer molecule to at least the blood contacting surface with a wet chemistry treatment including an aqueous solution having a cationic polymer with a cross linker, followed by adding a negatively charged spacer molecule to at least the blood contacting surface with a wet chemistry treatment including an aqueous solution having a anionic polymer, and followed by a step of adding a second positively charged spacer molecule to at least the blood contacting surface with a wet chemistry treatment including an aqueous solution having a cationic polymer.

5. The method of noise reduction in a blood panel testing platform for clinical application according to claim 4 wherein at least one of the steps of adding a positively charged spacer molecule to at least the blood contacting surface with a wet chemistry treatment including an aqueous solution having a cationic polymer utilizes PEI as the spacer molecule.

6. The method of noise reduction in a blood panel testing platform for clinical application according to claim 5 wherein by adding a negatively charged spacer molecule to at least the blood contacting surface with a wet chemistry treatment including an aqueous solution having an anionic polymer wherein dextran sulfate is utilized as the negatively charges spacer.

7. The method of noise reduction in a blood panel testing platform for clinical application according to claim 4 wherein the step of enhancing at least the blood contacting surface with a wet chemistry treatment including an aqueous solution having a strong oxidizing agent includes ammonium persulfate.

8. The method of noise reduction in a blood panel testing platform for clinical application according to claim 1 further including quality control steps of staining sacrificial inline tubing sections before wet chemistry treatments and after at least one wet chemistry treatment.

9. The method of noise reduction in a blood panel testing platform for clinical application according to claim 1 further including a drying step after the step of covalently immobilizing heparin to at least the blood contacting surface with a wet chemistry treatment including heparin, wherein the drying step includes a pressured gas flush.

10. The method of manufacturing a medical product having an engineered heparin bioactive matrix for clinical application according to claim 1 wherein the step of activating a blood contacting surface of at least one component of a medical device includes a plasma treatment.

11. A blood panel testing platform configured for noise reduction wherein the blood contacting surfaces of the blood panel testing platform have been activated and have been subsequently subject to wet chemistry treatment including
i) enhancing the blood contacting surface of the blood panel testing platform with a wet chemistry treatment including an aqueous solution having a strong oxidizing agent;
ii) adding a positively charged spacer molecule to the blood contacting surface with a wet chemistry treatment including an aqueous solution having a cationic polymer; and
iii) covalently immobilizing heparin to the blood contacting surface of the blood panel testing platform with a wet chemistry treatment including heparin.

12. The blood panel testing platform configured for noise reduction according to claim 11 wherein the spacer molecule is PEI.

13. The blood panel testing platform configured for noise reduction according to claim 11 wherein the step of adding a positively charged spacer molecule to at least the blood contacting surface with a wet chemistry treatment including an aqueous solution having a cationic polymer with a cross linker, followed by adding a negatively charged spacer molecule to at least the blood contacting surface with a wet chemistry treatment including an aqueous solution having a anionic polymer, and followed by a step of adding a second positively charged spacer molecule to at least the blood contacting surface with a wet chemistry treatment including an aqueous solution having a cationic polymer.

14. A method of noise reduction in a blood panel testing comprising the steps of:
Drawing blood samples from a patient;
Testing blood samples within a blood analyzer configured for noise reduction wherein the blood contacting surfaces of the blood panel testing platform have been activated and have been subsequently subject to wet chemistry treatment including
i) enhancing the blood contacting surface of the blood analyzer with a wet chemistry treatment including an aqueous solution having a strong oxidizing agent;
ii) adding a positively charged spacer molecule to the blood contacting surface with a wet chemistry treatment including an aqueous solution having a cationic polymer; and
iii) covalently immobilizing heparin to the blood contacting surface of the blood analyzer with a wet chemistry treatment including heparin.

15. The method of noise reduction in a blood panel testing according to claim 14 wherein the step of Drawing blood samples from a patient utilize blood receiving components treated for noise redection.

16. The method of noise reduction in a blood panel testing according to claim 15 wherein the step of enhancing at least the blood contacting surface with a wet chemistry treatment including an aqueous solution having a strong oxidizing agent includes ammonium persulfate.

17. The method of noise reduction in a blood panel testing according to claim 16 wherein the step of adding a positively charged spacer molecule to at least the blood contacting surface with a wet chemistry treatment including an aqueous solution having a cationic polymer utilizes PEI as the spacer molecule.

18. The method of noise reduction in a blood panel testing according to claim 15 wherein the step of adding a positively charged spacer molecule to at least the blood contacting surface with a wet chemistry treatment including an aqueous solution having a cationic polymer with a cross linker, followed by adding a negatively charged spacer molecule to at least the blood contacting surface with a wet chemistry treatment including an aqueous solution having a anionic polymer, and followed by a step of adding a second positively charged spacer molecule to at least the blood contacting surface with a wet chemistry treatment including an aqueous solution having a cationic polymer.

19. The method of noise reduction in a blood panel testing according to claim 18 wherein at least one of the steps of adding a positively charged spacer molecule to at least the blood contacting surface with a wet chemistry treatment including an aqueous solution having a cationic polymer utilizes PEI as the spacer molecule.

20. The method of noise reduction in a blood panel testing according to claim 19 wherein by adding a negatively charged spacer molecule to at least the blood contacting surface with a wet chemistry treatment including an aqueous solution having an anionic polymer wherein dextran sulfate is utilized as the negatively charges spacer.
